(19) United States
(12) Patent Application Publication
(10) Pub. No.: US 2019/0270093 A1
(43) Pub. Date: Sep. 5, 2019

(54) MAGNETIC TOOL AND METHOD OF COLLECTING MAGNETIC PARTICLES USING SAME

(71) Applicant: The University of Western Australia, Crawley, Western Australia (AU)

(72) Inventors: Robert Woodward, Balcatta, Western Australia (AU); Timothy St Pierre, South Fremantle, Western Australia (AU); Renata Russo Frasca Candido, South Fremantle, Western Australia (AU)

(21) Appl. No.: 16/334,065

(22) PCT Filed: Sep. 15, 2017
(86) PCT No.: PCT/AU2017/051010

§ 371 Date: Mar. 18, 2019

(30) Foreign Application Priority Data
Sep. 16, 2016 (AU) 2016903730

(51) Int. Cl. B03C 1/033; B03C 1/01; B03C 1/28; G01N 33/543

(52) U.S. Cl. CPC B03C 1/0332; B03C 1/01; B03C 2201/26; G01N 33/54326; B03C 2201/18; B03C 1/286

(57) ABSTRACT

A magnetic tool (10) has a probe (12) made of a material having very high permeability and a magnetic field source (14). The magnetic tool (10) has a body (18) which supports the probe (12) and the magnetic field source (14). The source (14) can be moved move within the body 18/cylinder (20). The probe (12) is in the form of a mu-metal needle formed with a sharpened point at its tip (16) and having an opposite end (26) embedded or otherwise fixed in the body (18). Therefore the tip (16) is at a fixed distance from the end of the body (18). The tool (10) is arranged to vary magnetic coupling between the magnetic field source (14) and the probe (12) between a maximum when the source (14) contacts the end (26) of the probe (12) and a minimum when the source is moved away from the probe (16).

### TECHNICAL FIELD

A magnetic tool is disclosed together with a method of collecting magnetic particles using the magnetic tool. The magnetic particles may be of a microscopic scale. The magnetic tool and method may be used for example for detecting parasite eggs in mammalian biological material including waste material such as urine and faecal matter.

### BACKGROUND ART

For several parasitic diseases such as schistosomiasis, diagnosis requires identification of parasite eggs in a sample of urine or faeces. For example one common method for detecting the parasite eggs for humans is inspection of a faecal smear using an optical microscope. Eggs are typically of the order of 100 microns in size and so are readily identified using standard optical microscopes used in pathology laboratories.

A major drawback of the standard faecal smear test is that only a small sample of faecal matter (typically between 50 and 60 milligrams) is assessed and as such the likelihood of a false negative test (i.e. no detection of eggs when eggs are present in the faecal matter of the patient) is very high when the egg burden is low. In an attempt to overcome this problem, Teixeira and colleagues developed a method of examining larger quantities of faecal matter to increase the probability of finding eggs if they are present. [Teixeira, C. F. et al., Detection of Schistosoma mansoni Eggs in Feces through their Interaction with Paramagnetic Beads in a Magnetic Field. PLoS Negl Trop Dis, 2007. 1(2): p. e73.]

The method involves using up to 30 g of faecal matter suspended in tap water. Several filtration steps followed by resuspension of sediment are used with a final step involving the use of magnetic particles. Magnetic particles are mixed with the sediment and subsequently a permanent magnet is used to attract the particles to the side of a microcentrifuge tube. By virtue of the affinity of schistosome eggs for the magnetic particles, the sediment attracted to the side of the microcentrifuge tube is enriched in eggs. An examination of this magnetic fraction of sediment with an optical microscope is the final step of this process known as the Helmintex technique. The optical examination can take up to several hours per sample.

The above references to the background art do not constitute an admission that the art forms a part of the common general knowledge of a person of ordinary skill in the art. Further, the above references are not intended to limit the application of the system, method and equipment as disclosed herein. Specifically embodiments of the disclosed magnetic tool and method of collecting magnetic particles are not limited to detecting parasite eggs in faecal matter, but extends more widely to include, but is not limited to, detecting parasite eggs in mammalian biological material including waste material, blood and other tissue.

### SUMMARY OF THE DISCLOSURE

In one aspect there is disclosed a magnetic tool comprising:
a body having a first end;
a probe supported at the first end of the body and made of a material having very high magnetic permeability, the probe having a tip at a fixed distance from the first end; and
a magnetic field source;
the tool being arranged to vary magnetic coupling between the magnetic field source and the probe between a maximum and a minimum wherein at maximum coupling magnetic flux from the magnetic field source couples with the probe to create a high magnetic field gradient at the tip of the probe and, at a minimum coupling, the magnetic field and field gradient at the tip of the probe is substantially zero or otherwise insufficient to attract magnetic particles.

In one embodiment the magnetic tool comprises a control mechanism capable of controlling the degree of magnetic coupling between the magnetic field source and the probe between the maximum and the minimum.

In one embodiment the control mechanism is capable of varying physical spacing between the magnetic field source and the probe wherein when the magnetic coupling is at a maximum the physical spacing between the magnetic field source and the probe is at a minimum.

In one embodiment the minimum spacing is zero such that the magnetic field source is in physical contact with the probe.

In one embodiment the magnetic tool comprises a body supporting the probe and the magnetic field source wherein the magnetic field source is movable relative to the probe by operation of the control mechanism to vary the degree of magnetic coupling between the magnetic field source and the probe.

In one embodiment the body has a tubular portion with a first end and a second end, wherein the probe is supported at the first end and the magnetic field source is able to be traversed along the body toward and away from the first end by the control mechanism.

In one embodiment the body has an opening at the second end through which the magnetic field source can be withdrawn from the body.

In one embodiment the control mechanism is coupled to the magnetic field source and capable of being manipulated by a user to vary the spacing between the magnetic field source and the probe.

In one embodiment the magnetic tool comprises the control mechanism being magnetically coupled to the magnetic field source.

In one embodiment the control mechanism comprises a magnetically soft iron member.

In one embodiment the probe is made from mu-metal.

In one embodiment the magnetic field source comprises a permanent magnet.

In one embodiment the permanent magnet is a rare earth magnet.

In another aspect there is disclosed a method of collecting magnetic particles carried in a liquid or slurry comprising:
inserting a probe into the liquid or slurry;
generating a magnetic field having a high magnetic field gradient emanating from the probe wherein magnetic particles in the liquid or slurry are attracted to and magnetically coupled to the probe; and
withdrawing the probe from the liquid or slurry.

In one embodiment the method comprises reducing the strength of the magnetic field subsequent to withdrawing the probe to facilitate release of the magnetic particles from the probe.

In one embodiment reducing the magnetic field comprises reducing magnetic flux coupling between a magnetic field source used to generate the magnetic field and the probe.

In one embodiment reducing the magnetic field coupling comprises moving the magnetic field source away from an end of the probe.

In one embodiment the method comprises mixing the magnetic particles in a liquid or slurry containing biological material having an affinity for the magnetic particles wherein the biological material is capable of being carried through the liquid or slurry by the magnetic particles to the probe.

In one embodiment the biological material comprises parasite eggs.

In one embodiment inserting the probe comprises inserting the probe of the magnetic tool according to the first aspect.

In a third aspect there is disclosed a method of detecting parasite eggs in faecal matter comprising:
mixing a plurality of magnetic particles in a fluid suspension containing a quantity of faecal matter;
immersing into the suspension a probe from which a magnetic field having a high magnetic field gradient emanates for a period of time sufficient to enable magnetic particles in the suspension to be magnetically coupled to the probe;
withdrawing the probe from the suspension;
optically inspecting the magnetic particles withdrawn from the slurry by the probe for parasite eggs from the fluid suspension.

In one embodiment withdrawing the probe comprises withdrawing the probe with a single droplet of the liquid, fluid or suspension adhered by surface tension to a tip of the probe.

### BRIEF DESCRIPTION OF THE DRAWINGS

Notwithstanding any other forms which may fall within the scope of the system, method and apparatus as set forth in the Summary, specific embodiments will not be described, by way of example only, with reference to the accompanying drawings in which:

FIG. 1a is a photograph of S. mansoni eggs and a scale bar of a length of approximately 100 microns;

FIG. 1b is a photograph of S. japonicum eggs and a scale bar of the length of approximately 100 microns;

FIG. 2 is a schematic representation of an embodiment of the disclosed magnetic tool in a state or configuration in which magnetic field gradient of a magnetic field emanating from a probe of the tool is at a maximum, this may be considered to be a magnetised or ON state;

FIG. 3 is a schematic representation of the magnetic tool shown in FIG. 2 in a state or configuration in which magnetic field gradient of a magnetic field emanating from a probe of the tool is at a minimum, this may be considered to be a demagnetised or OFF state;

FIG. 4 is a sequence of frames at relative time points of 0, 1, 2, and 3 seconds from a video recording of the field of view through an optical microscope focussed on the tip of the magnetized probe (left) submerged in a suspension of schistosome eggs that had been incubated with 4 micron sized magnetic microspheres. The arrows indicate approximate velocity vectors with the direction of the arrow indicating the direction of travel of the egg and the length of the arrow being proportional to the approximate speed of the egg.

FIG. 5 is a bar graph showing the results of tests performed using embodiments of the disclosed system, method and apparatus.

### DETAILED DESCRIPTION OF SPECIFIC EMBODIMENTS

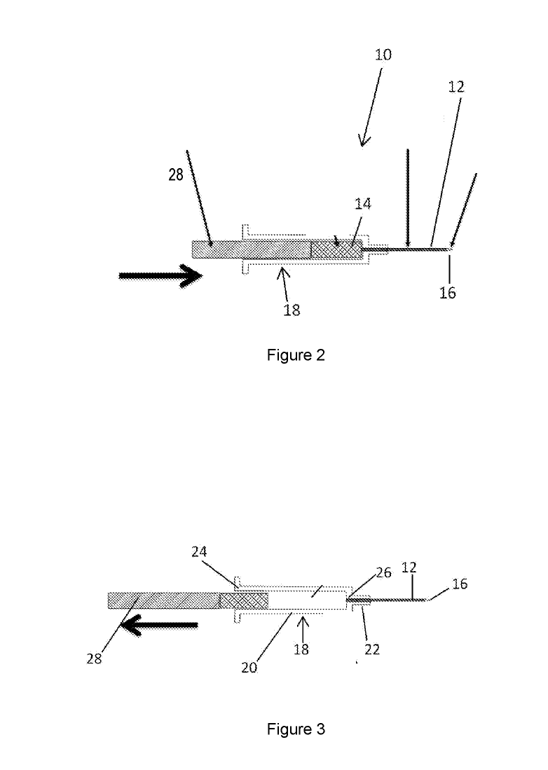

FIGS. 2 and 3 depict an embodiment of the magnetic tool 10 in respective operational states. The essence of the magnetic tool 10 is a combination of a probe 12 made of a material having very high permeability and a magnetic field source 14. The tool 10 is arranged to vary magnetic coupling between the magnetic field source 14 and the probe 12 between a maximum and a minimum. At maximum flux coupling the magnetic flux from the magnetic field source 14 couples with the probe to create a high magnetic field gradient at a tip 16 of the probe 12. This may be considered as the magnetised or ON state of the tool 10. When the magnetic flux coupling is at a minimum the magnetic field gradient at the tip 16 of the probe is substantially zero or otherwise insufficient to attract magnetic particles. This may be considered as the demagnetised or OFF state of the tool 10.

The magnetic tool 10 has a body 18 which supports the probe 12 and the magnetic field source 14. The body 18 may conveniently be formed from a plastics material and comprise a cylinder 20 having a coaxial stub 22 at one end. In one example the body may have a length of 50 mm-80 mm. An opposite end 24 of the cylinder portion 20 is open.

The magnetic field source 14 may for example be in the form of a rare earth magnet such as but not limited to a NdFeB rare earth magnet. The source 14 is relatively configured so that it may slide or move within the body 18/cylinder 20. Conveniently there is a loose fit between the source 14 and the inside of the cylinder 18. The probe 12 is in the form of a mu-metal needle formed with a sharpened point at its tip 16 and having an opposite end 26 embedded or otherwise fixed in the stub 22. Therefore the tip 16 is at a fixed distance from the end of the body 18, i.e. the stub 22. Having the tip 16 spaced from the end of the body 18 ensures there is no interference with the collection of a single droplet at the tip from other components of the tool 10. Such interference may arise for example with a tool having a permanent magnetic probe (unlike the presently disclosed tool) and a shielding sleeve that can slide up and down along the probe to reduce the magnetic field emanated from the probe. When the sleeve is near or adjacent the tip of the probe there is the risk that the sleeve will hamper or prevent the formation of a single droplet due to surface tension and capillary action between the interior of the sleeve and the exterior probe. In the event that capillary action causes ingress of liquid in between the sleeve and the probe transferring the liquid onto a slide for examination by microscope becomes problematic.

The sharpened point at the tip 16 creates a point attractor rather than a large area attractor which concentrates the particles into a single droplet volume for immediate microscopic examination. As an example the tip width may be in the order of tenths of a millimeter and more over less than 0.5 mm.

Once inserted into the cylinder 20 the magnetic field source 14 is attracted to the probe 12 due to the high magnetic permeability of the probe 12. In the absence of any counteracting force the magnetic field source is able to be located at a minimum spacing, which may include zero spacing (i.e. physical contact), to the end 26 of the probe 12. The probe 12 is magnetised and magnetic flux coupling between the source 14 and the probe 12 is at a maximum. Magnetic flux emanating from the tip 16 creates a point like source of high magnetic field gradient to which particles having high magnetic susceptibility are attracted.

Variation in magnetic flux coupling is achieved in this embodiment by varying the spacing between the magnetic field source 14 and the probe 12. A control mechanism 28 which in this embodiment is in the form of a magnetically soft iron rod may be used to selectively vary the spacing between the source 14 and the probe 12 thereby controlling the degree of flux coupling. By forming the control mechanism 28 as a magnetically soft iron rod, the control mechanism 28 is itself magnetically coupled to the magnetic field source 14. Additionally the magnetically soft iron rod acts to conduct magnetic flux away from the probe 12. The control mechanism 28 may be used to fully withdraw the magnetic field source 14 from the body 18/cylinder 22 to demagnetise the probe 12, placing the magnetic tool 10 in the demagnetised or OFF state and enabling the release of any magnetised material from the probe 12.

This embodiment of the tool 10 is configured so that when in the ON or magnetised state any magnetic flux from magnetic field source 14 which is not coupled into the probe 12 has miniscule strength and flux gradient at the tip 16. The magnetic field and gradient at the tip is overwhelmingly dominated by flux coupled directly into the probe 12. This follows from the inverse relationship of magnetic flux strength with distance from the source. The configuring of the tool to have the operational effect arises from the distance between the tip 16 and the source 14 arising from the length of the probe, and the narrowness of the probe and in particular the tip in comparison to the source 14. This provides suitability for at least the collection of small microscopic magnetic particles or non-magnetic microscopic particles to which microscopic magnetic particles are attached or otherwise adhered as exemplified below.

Thus embodiments of the disclosed tool 10 and associated method can be used to detect whether parasite eggs or other biological or non-biological particles (which may be conveniently referred to as “target particles”) are contained within a biological material or carrier material such as urine or faeces. This requires that the parasite eggs or other particles are in effect conditioned to be attracted by magnetic field by mixing with magnetic particles and subsequently attaching to or otherwise binding with the magnetic particles. Thus in a general sense the disclosed tool and method provide for the detection of magnetic particles which includes inherently non-magnetic particles that are made to be magnetic by attachment to or binding with magnetic particles. Naturally the disclosed method will not result in the detection of parasite eggs if such eggs are not present in the biological material (e.g. urine, faeces or other tissue) being sampled.

The results of tests and experiments using the tool 10 and associated method will now be described.

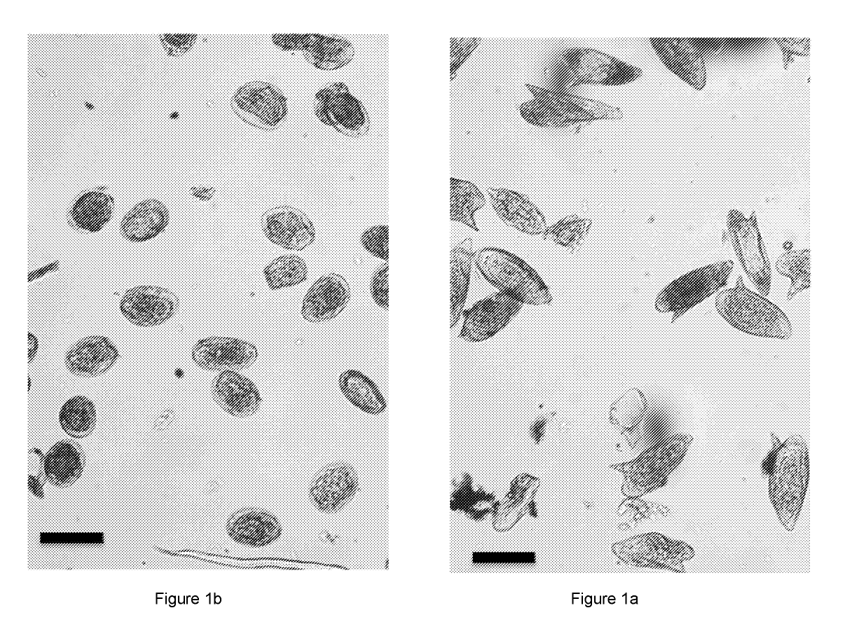

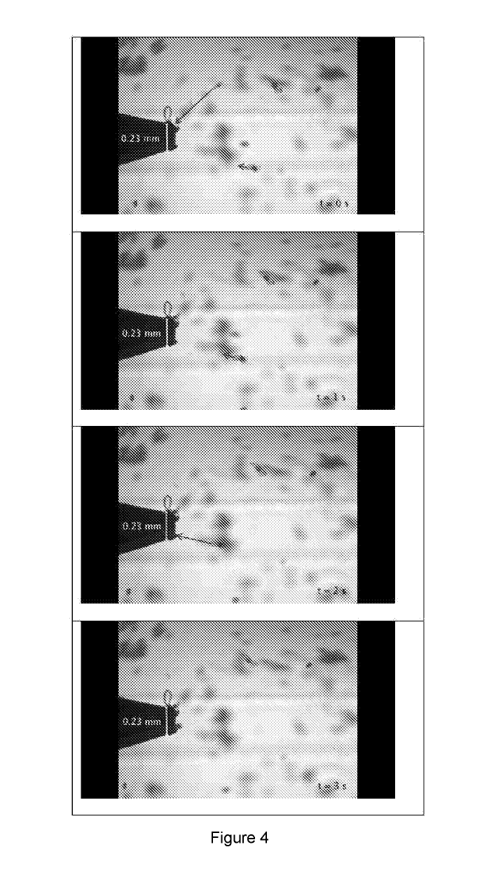

One test of the magnetic tool 10, described here with reference to FIG. 4, involved observing the behaviour of schistosome eggs as shown for example in FIGS. 1a and 1b suspended in normal saline with magnetic microspheres in the vicinity of the tip of the magnetized mu-metal rod.

Schistosome eggs were incubated with 4 μm diameter magnetic microspheres for 30 mins with gentle shaking. The suspension of eggs was then transferred to a shallow Perspex trough in which the mu-metal probe 12 was positioned. The magnetic tool 10 was placed in its magnetised or ON state with the magnetic field source 14 as close as possible to the probe 12, so that a magnetic field with the highest possible gradient emanates from the tip 16. An optical microscope focussed on the tip 16 of the probe 12 was then used to observe the behaviour of the schistosome eggs in the vicinity of the tip 16.

FIG. 4 shows frames from video footage of the microscope field of view over a period of three seconds. Schistosome eggs can be seen accelerating towards the tip 16 of the probe 12. The arrows in FIG. 4 represent approximate velocity vectors of the eggs at each time point. The direction of the arrow is the direction of travel and the length of the arrow is proportional to the speed of the egg. Observations from this test indicated that that an approximate radius of attraction of about 3 mm around the tip 16 was apparent.

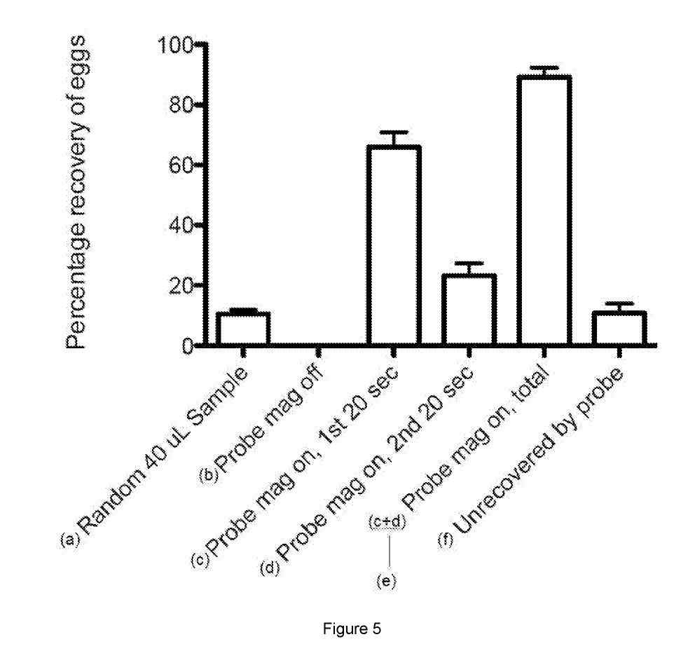

The following further test was carried out to identify the potential of the tool 10 to retrieve eggs from a suspension. In this study, no faecal matter was used. The results of this test are described with reference to FIG. 5.

1. Four microcentrifuge tubes containing different numbers of eggs (224+/−SD 85) were topped up with tap water to 500 μL. Into each microtube, 1 μL of iron oxide super-paramagnetic particle suspension (50 mg/mL in distilled water—BioMag BM547—Bangs Laboratories) was added.
2. The microcentrifuge tubes were agitated in a homogenizer for 30 minutes.
3. The microcentrifuge tube was shaken in a vortex mixer. A 40 μL sample (similar volume to a droplet that can be suspended from the probe tip 16 with surface tension) was taken from each microcentrifuge tube using a micropipette. The volume of fluid was placed on a microscope slide and the number of eggs counted. This is shown as bar (a) in FIG. 5. The bars in FIG. 5 depict the percentage recovery of eggs from the suspension.
4. The microcentrifuge tube was shaken in a vortex mixer. The tool 10 was placed in the de-magnetised or OFF state and the demagnetized probe 12 was used to stir the suspension for 20 seconds and then removed with a droplet adhered by surface tension to the tip 16. The droplet at the end of the tip 16 was then transferred to a glass microscope slide and the number of eggs counted. The egg count is shown as the bar (b) in FIG. 5. It will be seen that the bar (b) is a zero bar meaning that no eggs were attached to the demagnetised probe 12.
5. The microcentrifuge tube was shaken in a vortex mixer. The magnetic field source 14 was next positioned adjacent to the probe 16 using the control mechanism 28 thereby placing the tool 10 in the magnetised or ON state. The resultant magnetized probe 12 was used to stir the suspension for 20 seconds and then removed carrying a single droplet. The probe 12 was then demagnetized by withdrawing the magnetic field source 14 from the body 18 and the droplet at the end of the tip 16 was transferred to a glass microscope slide and the number of eggs counted. The egg count is shown as bar (c) in FIG. 5.
6. The microcentrifuge tube was shaken in a vortex mixer. The magnetized probe 12 was used to stir the suspension for another 20 seconds and then removed carrying a single droplet. The probe 12 was then demagnetized and the droplet at the end of the tip 16 was transferred to a glass microscope slide and the number of eggs counted. The egg count is shown as bar (d) in FIG. 5.
7. The bar (e) on FIG. 5 shows the sum of egg count of bars (c) and (d).
8. The eggs were allowed to settle in the microcentrifuge tube. A micropipette was then used to extract the egg sediment and transfer to a glass microscope slide and the number of eggs counted. These represented eggs not retrieved by the previous samplings. The number of unrecovered eggs is shown as bar (f) in FIG. 5.

The results of this test indicate that the magnetic tool 10 has a very high efficiency of extracting eggs from an aqueous suspension by concentrating them into an approximately 40-μL droplet attached to the tip 16 of the probe 12 by surface tension.

A further test was made of the tool 10 to gauge whether it can be used to enhance the performance of the previously described Helmintex method.

This test was carried out on samples of human faeces seeded with a known number of schistosoma eggs.

Six 30-g samples of human faeces were each seeded with 110±10 S. mansoni eggs.

The following steps were then used to process the samples for inspection for eggs with an optical microscope.

Each faecal sample was mixed with ethanol 70% for 30 minutes and then with ethanol 70%+Tween-20 10% (1:1) and left to rest for 30 minutes.

The mixture was passed through a 1-mm gauze mesh and left to sediment for one hour.

The supernatant was discarded and sediment resuspended four times until the supernatant was clear.

The sediment was then passed through a 150-μm and 45-μm mesh.

The sediment was left to rest for 30 minutes.

The supernatant was discarded and the sediment was placed into a 15-mL Falcon tube and tap water was added until the Falcon tube content reached 10 mL.

3 mL of ethyl acetate was added into the Falcon tube.

The Falcon tube was centrifuged for 10 minutes at 600 g.

The supernatant was discarded and the sediment was placed into a 1.5-mL microcentrifuge tube.

Tap water was added to top up the microcentrifuge tube to 1.0 mL.

Nineteen microlitres of super-paramagnetic particle suspension (BioMag® BM 547—Bangs Laboratories) was added to the microcentrifuge tube.

The microcentrifuge tube contents were homogenized in the microcentrifuge tube for 30 minutes.

The microcentrifuge tube was placed against a permanent magnet (using a BioMag® multi-6 microcentrifuge tube separator—Bangs Laboratories Inc) for 3 minutes. After 3 minutes, the microcentrifuge tube was inverted while still in contact with the magnet to pour out the contents. Material that was retained in the tube via the magnetic forces was then resuspended in 100 microliters of 0.9% saline solution.

The magnetized probe 12 of tool 10 was used to stir the suspension in the microcentrifuge tube for 20 s. The probe 12 was removed and the droplet retained at the tip 16 of the probe 12 was washed off the probe onto a glass microscope slide using 40 microlitres of tap water with the tool 10 and thus the probe 12 in the demagnetized state. A cover slip was then placed over the droplet in preparation for examination by optical microscopy.

The above step was repeated to produce a second sample mounted on a glass slide.

Each glass slide was inspected by optical microscope and the number of schistosome eggs was counted.

The following results were obtained:

| Faecal Sample Number | Number of Eggs Detected |
|---|---|
| 1 | 3 |
| 2 | 3 |
| 3 | 4 |
| 4 | 7 |
| 5 | 1* |
| 6 | 4 |

*There was a sample spillage during the sedimentation step for this sample and some eggs may have been lost.

The average time taken to examine the two slides for a sample was 16 minutes.

### CONCLUSION

At an egg burden of approximately 3.7 eggs per gram of faeces, the use of the tool 10 in the Helmintex method results in 100% sensitivity with a total slide examination time of approximately 16 minutes as opposed to several hours for the standard Helmintex method.

In general terms in embodiments of the disclosed method may involve stirring, agitating or otherwise simply maintaining the probe 16 with the tool 10 in the ON state within a small volume of liquid/suspension for example, but not limited to, about or less than 2-3 ml, such as 1.5 ml; for a period of 5-30 seconds or any sub period such as 5-20 seconds or 5-10 seconds; then withdrawing the probe with a single droplet of liquid. The single droplet may typically have a volume in the order of about 40 μL. The droplet can be placed on a microscope slide, the tool 10 turned OFF, and the droplet washed off with an equivalent volume of water.

### Additional Experiments

The following reports data from experiments designed to assess:
(a) whether magnetic iron oxide particles bind to different types of parasite egg
(b) whether an embodiment of the disclosed tool and associated method can efficiently extract parasite eggs from aqueous suspension
(c) the risk of cross contamination of samples by reusing the tool.

The parasite eggs tested in these experiments comprise:
(a) Haemonchus contortus (nematode) eggs isolated from sheep faeces
(b) Fasciola hepatica (trematode) eggs (fixed in formalin) isolated from sheep faeces
(c) Schistosoma haematobium eggs (fixed in ethanol 70%) isolated from human urine.

1. Binding of Magnetic Particles with Eggs of Haemonchus contortus (Nematode)
A—Sheep faecal samples containing Haemonchus contortus eggs were donated from the Division of Agriculture Diagnostics and Laboratory Services of WA.
B—The eggs were isolated through a process of sieving and mixing with saturated salt solution.
C—8 microtubes were produced containing approximately 100 Haemonchus contortus eggs and 1 mL of tap water
D—1 microlitre of magnetic iron oxide particles was added to each microtube and homogenized for 30 minutes
E—The magnetic probe was inserted in each tube and stirred for 5-10 seconds twice
F—The material collected at the tip 16 of the probe 12 in the two attempts was analysed by optical microscopy on a glass slide and the number of eggs counted
G—The bottom of each tube was analysed for assessing the number of eggs that was not collected

| Microtube number | 1^{st} Attempt | 2^{nd} Attempt | Bottom of tube |
|---|---|---|---|
| 1 | 36 | 3 | 1 |
| 2 | 47 | 1 | 0 |
| 3 | 41 | 5 | 5 |
| 4 | 42 | 0 | 2 |
| 5 | 47 | 2 | 0 |
| 6 | 51 | 5 | 1 |
| 7 | 38 | 0 | 0 |
| 8 | 42 | 2 | 2 |

These results show that the eggs of Haemonchus contortus (a nematode) readily bind magnetic iron oxide particles in sufficient quantities to be readily concentrated and extracted from aqueous suspensions using the probe.

2. Binding of Magnetic Particles with Eggs of Fasciola hepatica (Trematode)
A—Samples containing isolated Fasciola hepatica eggs fixed in formalin were donated from the Division of Agriculture Diagnostics and Laboratory Services of WA.
B—9 microtubes were produced containing approximately 150 Fasciola hepatica eggs and 1 mL of tap water
C—1 microlitre of magnetic iron oxide particles was added to each microtube and homogenized for 30 minutes
D—The tool 10 with magnetised probe 12 was inserted in each tube and stirred for 5-10 seconds twice
E—The material collected at the tip 16 of the probe 12 in the two attempts was analysed by optical microscopy on a glass slide and the number of eggs counted
F—The bottom of each tube was analysed for assessing the number of eggs that was not collected

| Microtube number | 1^{st} Attempt | 2^{nd} Attempt | Bottom of tube |
|---|---|---|---|
| 1 | 128 | 3 | 0 |
| 2 | 113 | 12 | 0 |
| 3 | 130 | 2 | 5 |
| 4 | 111 | 13 | 0 |
| 5 | 138 | 0 | 0 |
| 6 | 128 | 2 | 1 |
| 7 | 131 | 0 | 0 |
| 8 | 131 | 1 | 0 |
| 9 | 139 | 0 | 0 |

These results show that the eggs of Fasciola hepatica (a trematode) readily bind magnetic iron oxide particles in sufficient quantities to be readily concentrated and extracted from aqueous suspensions using the tool 10.

3. Binding of Magnetic Particles with Eggs of Schistosoma haematobium (Trematode)
A—Samples containing isolated Schistosoma haematobium eggs fixed in ethanol 70% were donated by from the Liverpool School of Tropical Medicine.
B—6 microtubes were produced containing approximately 15 Schistosoma haematobium eggs and 1 mL of tap water
C—1 microlitre of magnetic iron oxide particles was added to each microtube and homogenized for 30 minutes
D—The tool 10 in the ON state, i.e. with magnetised probe 12 was inserted in each tube and stirred for 5-10 seconds twice
E—The material collected at the tip 16 of the probe 12 in the two attempts was analysed by optical microscopy on a glass slide and the number of eggs counted
F—The bottom of each tube was analysed for assessing the number of eggs that was not collected

| Microtube number | 1^{st} Attempt | 2^{nd} Attempt | Bottom of tube |
|---|---|---|---|
| 1 | 8 | 3 | 1 |
| 2 | 10 | 2 | 2 |
| 3 | 10 | 0 | 2 |
| 4 | 15 | 1 | 0 |
| 5 | 13 | 1 | 0 |
| 6 | 10 | 2 | 1 |

These results show that the eggs of Schistosoma haematobium (a trematode) readily bind magnetic iron oxide particles in sufficient quantities to be readily concentrated and extracted from aqueous suspensions using the tool 10.

4. Experiments Performed to Assess Cross Contamination Between Uses with the Tool 10
A—After every use with the tool 10, the tip 16 of the probe 12 was thoroughly cleaned with water and a piece of tissue paper
B—The tip 16 of the probe was then washed onto a glass slide and the material was analysed using optical microscopy for assessing the presence of eggs
C—This procedure was repeated 10 times
D—No eggs were found in any attempt

Whilst specific embodiments have been described it should be appreciated that the disclosed magnetic tool, method of collecting magnetic particles carried in a liquid or slurry; and method of detecting parasite eggs in biological matter may be embodied in many other forms. For example the control member 28 is described as being a magnetically soft iron rod which is magnetically coupled to the source 14. However the control member 28 could be in the form of a rod made from plastics or other materials such as wood or composite materials. Also while the magnetic field source is described as being a rare earth permanent magnet it may be in the form of an electromagnet. In that instance the flux coupling between the magnetic field source and the probe 12 can be electronically controlled by varying the current through electromagnet. In that embodiment the control mechanism may for example be a potentiometer of a power/current unit. It is also to be stressed that the use of the tool 10 and associated described methods are not limited in application to detecting or collecting biological material, and less so parasite eggs. Rather the tool 10 and associated methods can be used for detecting or collection any magnetic or magnetisable particles and other particles that can be carried thereby.

In the claims which follow, and in the preceding description, except where the context requires otherwise due to express language or necessary implication, the word “comprise” and variations such as “comprises” or “comprising” are used in an inclusive sense, ie to specify the presence of the stated features but not to preclude the presence or addition of further features in various embodiments of the magnetic tool, method of collecting magnetic particles carried in a liquid or slurry; and method of detecting parasite eggs in faecal matter, as disclosed herein.

1. A magnetic tool comprising:
a body having a first end;
a probe supported at the first end of the body and made of a material having very high magnetic permeability, the probe having a tip at a fixed distance from the first end; and
a magnetic field source;
the tool being arranged to vary magnetic coupling between the magnetic field source and the probe between a maximum and a minimum wherein at maximum coupling magnetic flux from the magnetic field source couples with the probe to create a high magnetic field gradient at the tip of the probe and, at a minimum coupling, the magnetic field and field gradient at the tip of the probe is substantially zero or otherwise insufficient to attract magnetic particles.

2. The magnetic tool according to claim 1 comprising a control mechanism capable of controlling the degree of magnetic coupling between the magnetic field source and the probe between the maximum and the minimum.

3. The magnetic tool according to claim 2 wherein the control mechanism is capable of varying physical spacing between the magnetic field source and the probe wherein when the magnetic coupling is at a maximum the physical spacing between the magnetic field source and the probe is at a minimum.

4. The magnetic tool according to claim 3 wherein the minimum spacing is zero such that the magnetic field source is in physical contact with the probe.

5. The magnetic tool according to claim 2 comprising a body supporting the probe and the magnetic field source wherein the magnetic field source is movable relative to the probe by operation of the control mechanism to vary the degree of magnetic coupling between the magnetic field source and the probe.

6. The magnetic tool according to claim 5 wherein the magnetic field source is able to be traversed along the body toward and away from the first end by the control mechanism.

7. The magnetic tool according to claim 6 wherein the body has an opening at a second end opposite the first end through which the magnetic field source can be withdrawn from the body.

8. The magnetic tool according to claim 4 wherein the control mechanism is coupled to the magnetic field source and capable of being manipulated by a user to vary the spacing between the magnetic field source and the probe.

9. The magnetic tool according to claim 8 wherein the control mechanism is magnetically coupled to the magnetic field source.

10. The magnetic tool according to claim 8 wherein control mechanism comprises a magnetically soft iron member.

11. The magnetic tool according to claim 1 wherein the probe is made from mu-metal.

12. The magnetic tool according to claim 1 wherein the magnetic field source comprises a permanent magnet.

13. The magnetic tool according to claim 12 wherein the permanent magnet is a rare earth magnet.

14. The magnetic tool according to claim 1 wherein the tip is formed with a sharpened point.

15. A method of collecting magnetic particles carried in a fluid suspension comprising:
inserting a probe into the fluid suspension;
generating a magnetic field having a high magnetic field gradient emanating from the probe wherein magnetic particles in the fluid suspension are attracted to and magnetically coupled to the probe; and
withdrawing the probe from the fluid suspension carrying a single drop of the fluid suspension.

16. The method according to claim 15 further comprising reducing the strength of the magnetic field subsequent to withdrawing the probe to facilitate release of the magnetic particles from the probe.

17. The method according to claim 16 wherein reducing the magnetic field comprises reducing magnetic flux coupling between a magnetic field source used to generate the magnetic field and the probe.

18. The method according to claim 17 wherein reducing the magnetic field coupling comprises moving the magnetic field source away from an end of the probe.

19. The method according to claim 15 comprising mixing the magnetic particles in a fluid suspension containing one or more biological particles having an affinity for the magnetic particles wherein the biological particles are capable of being carried through the fluid suspension by the magnetic particles to the probe.

20. The method according to claim 19 wherein the biological particles comprise parasite eggs.

21. The method according to claim 19 comprising forming the fluid suspension to contain a sample of biological material potentially containing the biological particles.

22. The method according to claim 15 wherein inserting the probe comprises inserting the probe of a magnetic tool comprising:
a body having a first end;
the probe supported at the first end of the body and made of a material having very high magnetic permeability, the probe having a tip at a fixed distance from the first end; and
a magnetic field source;
the tool being arranged to vary magnetic coupling between the magnetic field source and the probe between a maximum and a minimum wherein at maximum coupling magnetic flux from the magnetic field source couples with the probe to create a high magnetic field gradient at the tip of the probe and, at a minimum coupling, the magnetic field and field gradient at the tip of the probe is substantially zero or otherwise insufficient to attract magnetic particles.

23. A method of detecting parasite eggs in biological material comprising:
mixing a plurality of magnetic particles in a fluid suspension containing a quantity of biological material potentially containing parasite eggs;
immersing into the suspension a probe from which a magnetic field having a high magnetic field gradient emanates for a period of time sufficient to enable magnetic particles in the suspension to be magnetically coupled to the probe;
withdrawing the probe from the suspension with a single drop of fluid from the fluid suspension;
optically inspecting the single drop of fluid withdrawn from the fluid suspension for parasite eggs.
